(19) United States
(12) Patent Application Publication
(10) Pub. No.: US 2014/0056937 A1
(43) Pub. Date: Feb. 27, 2014

(54) WARM SENSATION RECOVERY LOTION AND METHOD FOR MANUFACTURING THE SAME

(71) Applicant: TENG-HUI WANG, TAINAN CITY (TW)

(72) Inventor: TENG-HUI WANG, TAINAN CITY (TW)

(73) Assignee: WELL MAX BEAUTY LAB CO., LTD., TAINAN CITY (TW)

(21) Appl. No.: 13/590,317

(22) Filed: Aug. 21, 2012

(51) Int. Cl. A61K 8/97; A61Q 19/00; A61K 8/99

(52) U.S. Cl. 424/195.16; 424/756; 424/750

(57) ABSTRACT

A kind or warm sensation recovery lotion and a method for manufacturing the same are revealed. Firstly mix a warming agent derived from vanillyl butyl ether (VBE) and a surface active agent derived from hydrogenated castor oil, zingiber officinale (ginger) root extract and plant extracts. Then add water as a solvent, mix and stir the solution to form the lotion. Thereby a connection between a function group of VBE causing a warm sensation and a sensory receptor for heat on cell membrane of a human nerve cell results in a warming effect on the skin. Moreover, the skin is recovered and moisturized by the plant extracts so that users have warming sensation and comfortable feelings.

### BACKGROUND OF THE INVENTION

1. Field of the Invention

The present invention relates to a kind of lotion and a method for manufacturing the same, especially to a kind of warm sensation recovery lotion that not only provides a warming effect on user's skin but also recovers and moisturizes the skin and a method for manufacturing the same.

2. Description of Related Art

Most of cosmetics available on the market that provides a warming sensation is produced by chili oil and chili extracts, ginger oil and ginger extracts, or pepper oil and pepper extracts. However, when these oil and extracts are added into hand lotion, they may irritate user's skin and cause a burning or other uncomfortable feeling.

Thus some heat-generating materials including far infrared mineral powder, zeolite powder and calcium chloride are provided to replace the above irritants to solve the problems of burning sensation and other uncomfortable feeling. Yet the far infrared mineral powder has poor heating effect in practice. Although the zeolite powder provides better effect, the manufacturing processes of the zeolite powder should keep dry otherwise the heat release for a given period is affected by water. This increases difficulties and restrictions on the manufacturing. Moreover, the hand lotion added with zeolite powder has a rough texture and this limits the user's acceptance. Furthermore, the calcium chloride also has similar problems caused by water during manufacturing process. And it's more irritant and hazardous in case of skin, contact and may cause damages to the skin.

### SUMMARY OF THE INVENTION

Therefore it is a primary object of the present invention to provide a kind, of warm sensation recovery lotion and a method for manufacturing the same that gives a warming sensation to the skin quickly and the warming sensation is maintained for a long period.

In order to achieve the above objects, a kind of warm sensation recovery lotion and a method for manufacturing the same are provided. The warm sensation recovery lotion mainly includes a warming agent derived from vanillyl butyl ether (VBE), a surface active agent derived from hydrogenated castor oil, zingiber officinale (ginger) root extract, and plant extracts. Then the mixture of the above ingredients is dissolved in pure water solvent.

The method for manufacturing kind of warm sensation recovery lotion includes following steps.

Step (A) prepare a solute of warm sensation lotion: mix and stir a warming agent derived from vanillyl butyl ether (VBE), a surface active agent derived from hydrogenated castor oil, and zingiber officinale (ginger) root extract in a mixing tank evenly to form the solute.

Step (B) mix to prepare a solution of warm sensation solution; add pure water solvent into the solute formed by the warming agent derived from vanillyl butyl ether, the surface active agent derived from hydrogenated castor oil, and the zingiber officinale (ginger) root extract to form the solution.

Step (C) emulsify: stir the solution formed by a mixture of the warming agent derived from vanillyl butyl ether, the surface active agent derived from hydrogenated castor oil, the zingiber officinale (ginger) root extract and the pure water solvent for homogenization and emulsion.

Step (D) add recovery materials: add plant extracts and the lemongrass oil into the mixture in and keep stirring the mixture.

Thereby the warm sensation recovery lotion of the present invention use VBE as the warming agent. By good connection between a function group of the VBE that causes a warm sensation and a sensory receptor for heat on cell membrane of a human nerve cell, a warming effect is produced on the skin and is maintained for a certain period. Thus the warm sensation recovery lotion can keep users warm against the cold weather.

### BRIEF DESCRIPTION OF THE DRAWINGS

The structure and the technical means adopted by the present invention to achieve the above and other objects can he best understood by referring to the following detailed description of the preferred embodiments and the accompanying drawings, wherein

FIG. 1 is a schematic drawing Showing composition of warm sensation and recovery lotion according to the present invention;

FIG. 2 is a schematic drawing showing ingredients of warm sensation lotion according to the present invention;

FIG. 3 is a schematic drawing showing ingredients of recovery lotion according to the present invention;

FIG. 4 is a schematic drawing showing a flow chart of manufacturing warm sensation lotion according to the present invention;

FIG. 5 is a schematic drawing showing a flow chart of manufacturing recovery lotion according to the present invention;

FIG. 6 is a schematic drawing showing a flow chart of manufacturing warm sensation and recovery lotion according to the present invention;

FIG. 7 is a schematic drawing showing an open calcium channel after a functional group causing a warm sensation connected to a sensory receptor for heat according to the present invention;

FIG. 8 is a schematic drawing showing transmission of an action potential triggered by calcium ions flowing, into nerve cells that causes depolarization of cell membrane according to the present invention;

FIG. 9 is a schematic drawing showing releasing of neurotransmitters;

FIG. 10 is a schematic drawing showing delivery of neurotransmitters to nerve cells of central nerve system.

### DETAILED DESCRIPTION OF THE PREFERRED EMBODIMENT

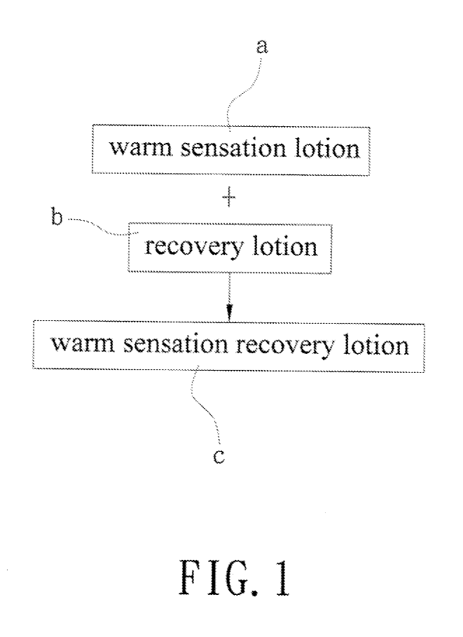

Refer to FIG. 1, a kind of warm sensation recovery lotion (c) of the present invention mainly includes warm sensation lotion (a) and recovery lotion (b), mixed with each other.

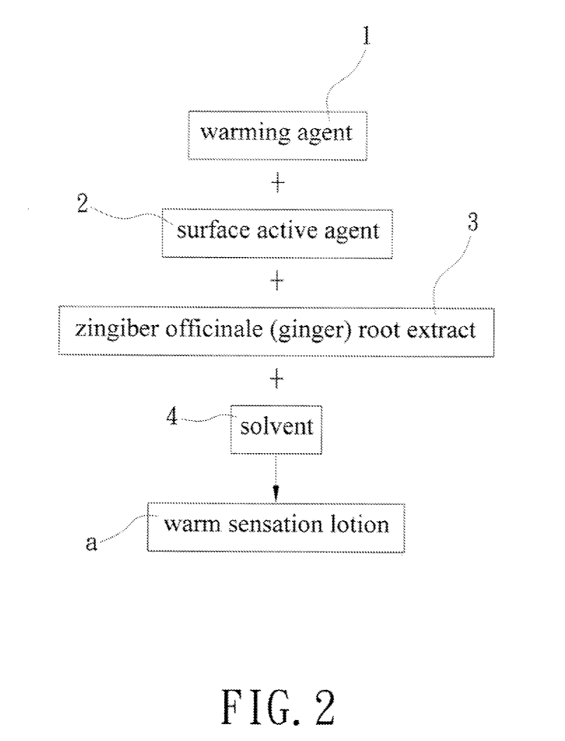

Refer to FIG. 2, the warm sensation lotion (a) consists of a warming agent 1 derived from vanillyl butyl ether (VBE), a surface active agent 2 derived from hydrogenated castor oil, zingiber officinale (ginger) root extract 3, and pure water used as solvent 4. The warming agent 1 derived from vanillyl butyl ether, the surface active agent 2 derived from hydrogenated castor oil and the zingiber officinale (ginger) root extract 3 are mixed and dissolved in the pure water solvent 4.

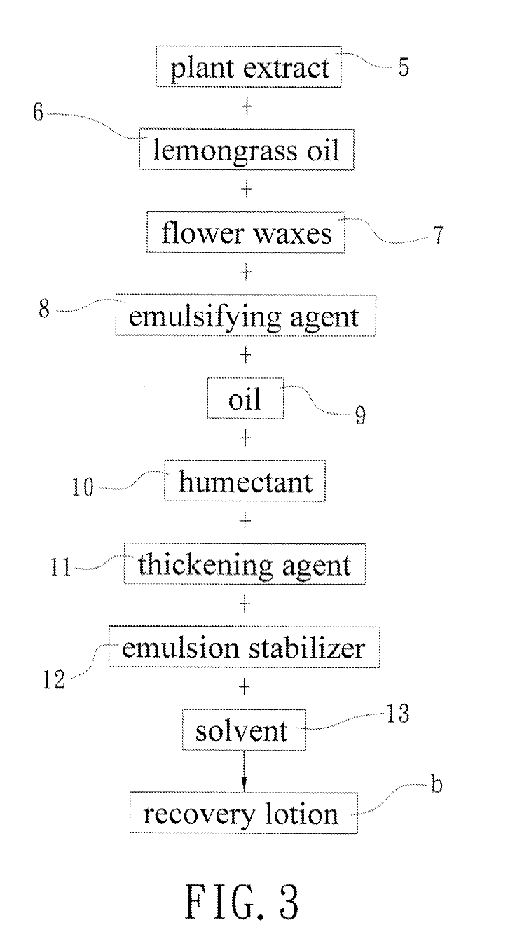

As to the recovery lotion (b), refer to FIG. 3, it is composed of plant extracts 5, lemongrass oil 6, mixed flower waxes 7, an emulsifying agent 8, oil 9, a humectant 10, a thickening agent 11, an emulsion stabilizer 12 and pure water as solvent 13. The plant extracts 5 include extracts of ruscus aculeatus, horse chestnut, centella asiatica, saccharomyces, marigold (calendula officinalis) and glycyrrhiza glabra.

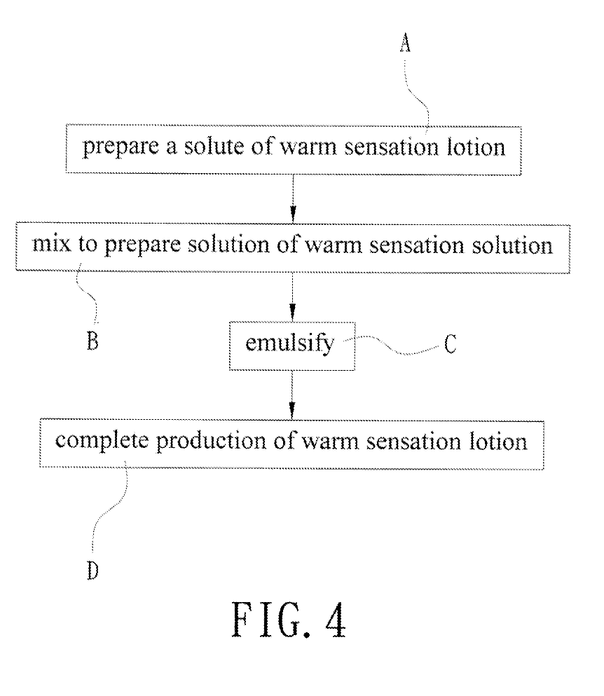

Refer to FIG. 4, while manufacturing he warm sensation solution (a), the steps are as following:

Step (A) prepare a solute of warm sensation lotion: stir and mix the warming agent 1 derived from vanillyl butyl ether, the surface active agent 2 derived from hydrogenated castor oil, and the zingiber officinale (ginger) root extract 3 in a mixing tank evenly at room temperature to form the solute;

Step (B) mix to prepare solution of warm sensation solution: add the pure water solvent 4 into the solute formed by the warming agent 1 derived from vanillyl butyl ether, the surface active agent 2 derived from hydrogenated castor oil, and the zingiber officinale (ginger) root extract 3 at room temperature to form a solution;

Step (C) emulsify: then at room temperature stir the solution formed by a mixture of the warming agent 1 derived from vanillyl butyl ether, the surface active agent 2 derived from hydrogenated castor oil, the zingiber officinale (ginger) root extract 3 and the pure water solvent 4 at 500-1000 rpm per min (rpm/min) for homogenization and emulsion;

Step (D) complete production of warm sensation lotion: after the warm sensation lotion (a) being homogenized, the warming agent 1 derived from vanillyl butyl ether therein is protected by micelles for extending its effects and stability.

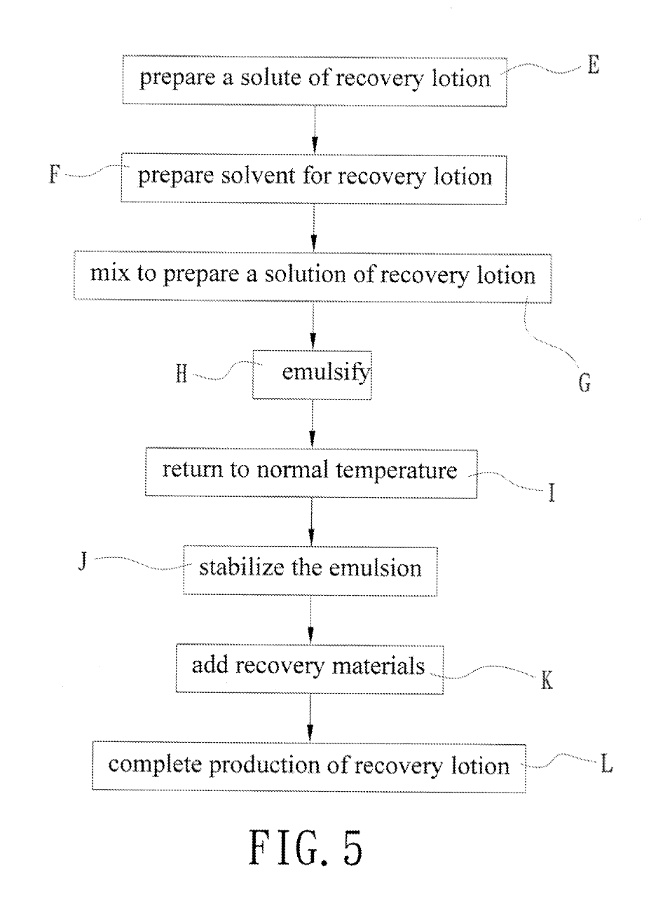

Refer to FIG. 5, a flow chart showing manufacturing of the recovery lotion (h) includes following steps.

Step (E): prepare a solute of recovery lotion: stir and mix the mixed flower waxes 7, the emulsifying agent 8, and the oil 9 in a mixing tank evenly and heat the mixture up to 60 degrees Celsius;

Step (F): prepare solvent for recovery lotion: stir and mix the humectant 10, the thickening agent 11 and the solvent (pure water) 13 in another mixing tank until the mixture has a certain consistency and heat the mixture up to 60 degrees Celsius;

Step (G): mix to prepare a solution of recovery lotion: slowly add the solvent for recovery lotion formed by the humectant 10, the thickening agent 11 and the solvent (pure water) 13 in the step F into the solute including the mixed flower waxes 7, the emulsifying agent 8, and the oil 9 formed in the step F to produce a solution of recovery lotion;

Step (H) emulsify: then stir the solution formed by the humectant 10, the thickening agent 11, the pure water solvent 13, the mixed flower waxes 7, the emulsifying agent 8, and the oil 9 at 600 rpm per min (rpm/min) for homogenization to form emulsion;

Step (I): return to normal temperature: keep stirring the emulsion at 600 rpm/min for returning to normal temperature;

Step (J): stabilize the emulsion: after the temperature of the emulsion reaching 30 degrees Celsius, add the emulsion stabilizer 12 into the emulsion and continue stirring the emulsion at 600 rpm/min;

Step (K): add recovery materials: add the plant extracts 5 and the lemongrass oil 6 into the emulsion in turn and keep stirring the mixture at 600 rpm/min for homogenization; and

Step (L): complete production of recovery lotion: the recovery lotion (h) reduces hypersensitivity of users' skin to the warming agent 1 derived from vanillyl butyl ether by the plant extracts 5 contained therein.

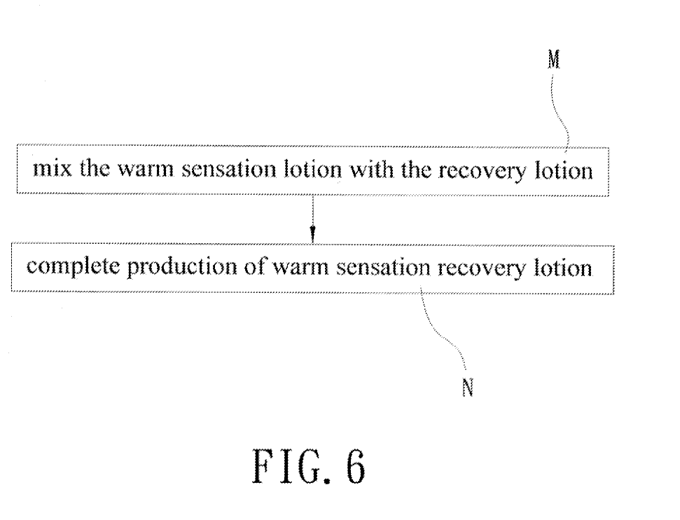

Refer to FIG. 6, prepare warm sensation recovery lotion (c) by following steps:

Step (M): mix the warm sensation lotion with the recovery lotion: add the warm sensation lotion (a) that is formed by the warming agent 1 derived from, vanillyl butyl ether, the surface active agent 2 derived from hydrogenated castor oil, the zingiber officinale (ginger) root extract 3 and the pure water solvent 4 into the recovery lotion (h) and then stir and mix the mixture evenly at 600 rpm/min for homogenization; and

Step (N): complete production of warm sensation recovery lotion.

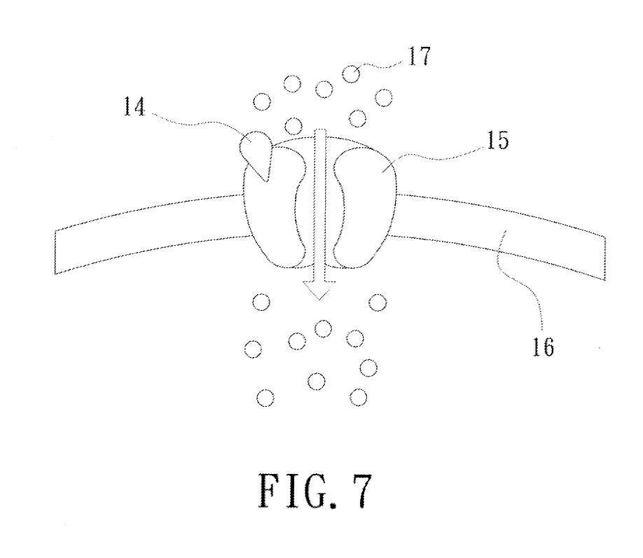

Thereby under cold weather or chill environment, refer to FIG. 7, users can apply the warm sensation recovery lotion (c) of the present invention to skin on their hands or other parts. At this moment, the warming agent 1 derived from vanillyl butyl ether contained in the warm sensation lotion (a) of the warm sensation recovery lotion (c) of the present invention includes a function group 14 causing a warm sensation that is connected to a sensory receptor for heat 15 on cell membrane of a human nerve cell 16 such as VR-1 that is activated by heat (Vanilloid Receptor-1, VR-1). After the function group 14 of the warming agent 1 derived from vanillyl butyl ether being connected to the sensory receptor for heat 15 (VR-1), a calcium channel of the human nerve cell 16 is opened to allow calcium ions 17 flowing into the nerve cell 16.

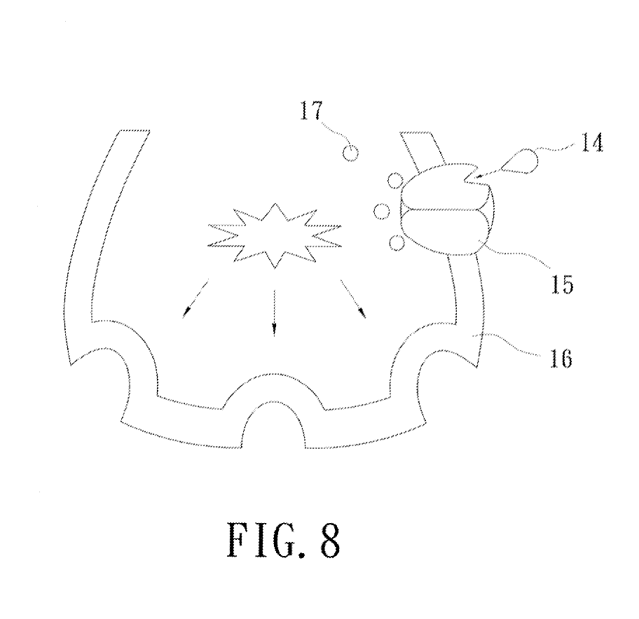

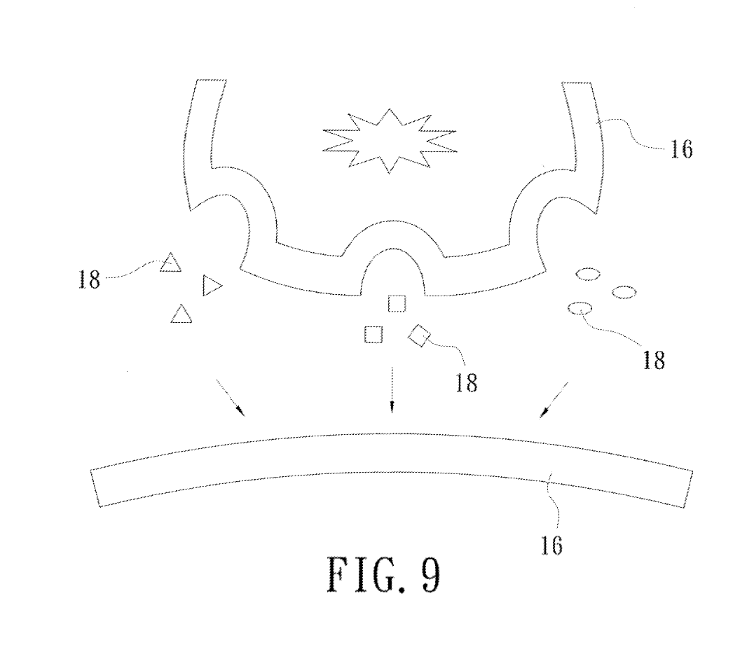

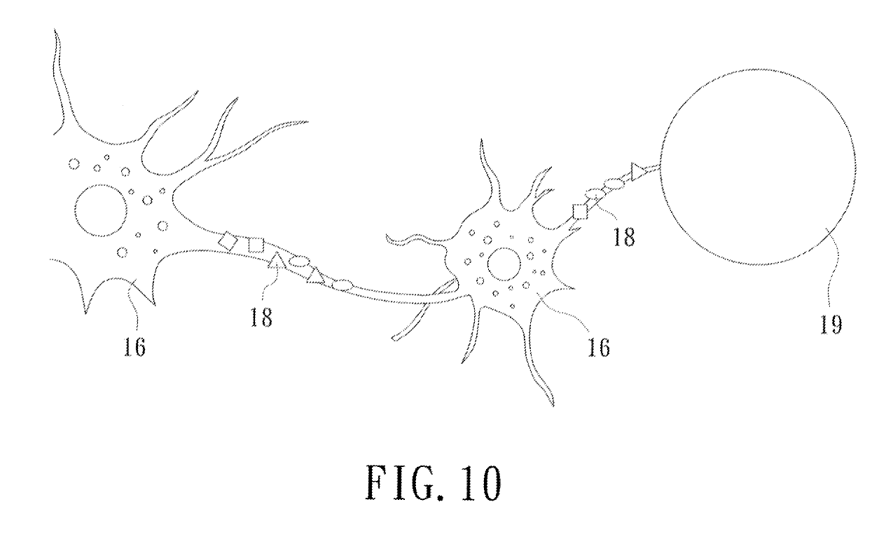

Next refer to FIG. 8, the calcium ions 17 flowing into the nerve cell 16 causes depolarization of cell membrane. An action potential is triggered when enough depolarization accumulates to bring the membrane potential up to threshold. Next the action potential (neuron impulse) is transmitted to another nerve cell 16 through an axon of the nerve cell 16. Refer to FIG. 9, when the action potential arrives to another nerve cell 16, it provokes release of several kinds of neurotransmitters 18 such as glutamate, adenosine triphosphate (ATP), and neuropeptides, etc. from this nerve cell 16. By means of these neurotransmitters 18, a central nerve system 19 receives a signal of a warm sensation to produce a heating effect on the body, as shown in FIG. 10.

Thereby once users apply the warm sensation recovery lotion (c) of the present invention to their skin, they have a warming sensation (a sensation of warming) to keep them warm in the chilly weather and the cold environment.

Moreover, the irritation possibly caused by the warming agent 1 derived from vanillyl butyl ether can be soothed or comforted by the plant extracts 5 contained in the recovery lotion (b) of the warm sensation recovery lotion (c). Thus the skin is soothed and comforted. As to the mixed flow waxes 7, they provide multiple effects on user's skin such as recovery, refreshing, whitening, and moisturizing. Moreover, lemon scent of the lemongrass oil 6 has functions of sterilization, deodorization and relaxation. Thereby the possible skin allergies can be prevented without inhibition of the warming sensation effect of the warm sensation recovery lotion (c) on the skin. Therefore the warm sensation recovery lotion (c) of the present invention provides users a warming sensation and long term usage without irritating sensation.

In summary, the present invention has following advantages:

1. The warm sensation recovery lotion of the present invention uses vanillyl butyl ether (VBE) as the warming agent. After the function group of the warming agent derived from vanillyl butyl ether being connected to the sensory receptor for heat on cell membrane of a human nerve cell, a warming sensation is produced on the skin quickly and the warming sensation is maintained for a long period. Thus the warm sensation recovery lotion can keep users warm against the cold weather.

2. The plant extracts and flower waxes included in the warm sensation recovery lotion of the present invention are used to recover and moisturize users' skin and further calm and balance the skin. Thus the skin discomfort is relieved and the lotion is gentle and safe to use.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalent

What is claimed is:

1. A kind of warm sensation recovery lotion comprising
at least one warming agent derived from vanillyl butyl ether (VBE),
at least one surface active agent derived from hydrogenated castor oil,
zingiber officinale (ginger) root extract,
at least one plant extract; and
at least one solvent.

2. The warm sensation recovery lotion, as claimed in claim 1, wherein the solvent is pure water.

3. The warm sensation recovery lotion as claimed in claim 1, wherein the plant extract is selected from a group consisting of ruscus aculeatus, horse chestnut, centella asiatica, saccharomyces, marigold (calendula officinalis) and glycyrrhiza glabra.

4. The warm sensation recovery lotion as claimed in claim 1, wherein the warm sensation recovery lotion further includes lemongrass oil and the lemongrass oil is mixed with the solvent.

5. The warm sensation recovery lotion as claimed in claim 4, wherein the warm sensation, recovery lotion, further includes flow waxes and the flow waxes is mixed with the solvent.

6. The warm sensation recovery lotion as claimed in claim 5, wherein the warm sensation recovery lotion further includes at least one ingredient selected from the group consisting of an emulsifying agent, oil, a humectant, a thickening agent, and an emulsion stabilizer and the ingredient is mixed with the solvent.

7. The warm sensation recovery lotion as claimed in claim 4, wherein the warm sensation recovery lotion further includes at least one ingredient selected from the group consisting of an emulsifying agent, oil, a humectant, a thickening agent, and an emulsion stabilizer and the ingredient is mixed with the solvent.

8. The warm sensation recovery lotion as claimed in claim 1, wherein the warm sensation recovery lotion further includes flow waxes and the flow waxes is mixed with the solvent.

9. The warm sensation recovery lotion as claimed in claim 1, wherein the warm sensation recovery lotion further includes at least one ingredient selected from the group consisting of an emulsifying agent, oil, a humectant, a thickening agent, and an emulsion stabilizer and the ingredient is mixed with the solvent.

10. A method for manufacturing a kind of warm sensation recovery lotion comprising the steps of
Step (A) preparing a solute of warm sensation lotion: mixing and stirring a warming agent derived from vanillyl butyl ether (VBE), a surface active agent derived from hydrogenated castor oil, and zingiber officinale (ginger) root extract in a mixing tank evenly to form the solute;
Step (B) mixing to prepare a solution of warm sensation solution; adding solvent into the solute formed by the warming agent derived from vanillyl butyl ether, the surface active agent derived from hydrogenated castor oil, and the zingiber officinale (ginger) root extract to produce the solution;
Step (C) emulsifying: stirring the solution formed by the warming agent derived from vanillyl butyl ether, the surface active agent derived from hydrogenated castor oil, the zingiber officinale (ginger) root extract and the solvent for homogenization and emulsion.

11. The method as claimed in claim 10, wherein the solvent is pure water.

12. The method as claimed in claim 10, wherein the method for manufacturing warm sensation recovery lotion further includes the steps of:
Step (D): preparing a solute of recovery lotion: stirring and mixing flower waxes, an emulsifying agent, and oil in a mixing tank evenly and heating mixture;
Step (E): preparing solvent for recovery lotion: stirring and mixing a humectant, a thickening agent and pure water in another mixing tank until mixture has a certain consistency and. heating the mixture;
Step (F): mixing to prepare a solution of recover lotion: slowly adding the solvent formed by the humectant, the thickening agent and the pure water into the solute formed by the flower waxes, the emulsifying agent, and the oil to produce a solution;
Step (G) emulsifying: stirring the solution formed by the humectant, the thickening agent, the pure water, the flower waxes, the emulsifying agent, and the oil for homogenization to form emulsion;
Step (H): returning to normal temperature: keeping stirring the emulsion to make temperature of the emulsion return to normal temperature;
Step (I): stabilizing the emulsion: after temperature of the emulsion returning to normal temperature, adding emulsion stabilizer into the emulsion and continuing stirring the emulsion;
Step (J): adding recovery materials: adding plant extracts and the lemongrass oil into the emulsion and keeping stirring the emulsion; and
Step (K): completing production of recovery lotion: adding the emulsified solution formed by the warming agent derived from vanillyl butyl ether, the surface active agent derived from hydrogenated castor oil, the zingiber officinale (ginger) root extract and the pure water into the emulsion and stirring until the emulsified solution and the emulsion are mixed with other evenly.
